(19) United States
(12) Patent Application Publication
(10) Pub. No.: US 2026/0115293 A1
(43) Pub. Date: Apr. 30, 2026

(54) METHODS AND COMPOSITIONS FOR PREVENTION AND TREATMENT OF ACUTE RADIATION SYNDROMES (ARS)

(71) Applicant: Maroon Biotech Corporation, Chicago, IL (US)

(72) Inventors: Kyle McCollum, Chicago, IL (US); Julia M. Radhakrishnan, Chicago, IL (US); Raphael C. Lee, Chicago, IL (US)

(21) Appl. No.: 19/006,521

(22) Filed: Dec. 31, 2024

Related U.S. Application Data
(60) Provisional application No. 63/617,495, filed on Jan. 4, 2024.

(51) Int. Cl. A61K 47/34; A61K 31/07; A61K 31/122; A61K 31/375; A61K 31/7016; A61K 31/7076; A61K 33/04; A61K 33/242; A61K 33/26; A61K 33/30; A61K 33/32; A61K 33/38; A61P 39/06

(52) U.S. Cl. CPC A61K 47/34; A61K 31/07; A61K 31/122; A61K 31/375; A61K 31/7016; A61K 31/7076; A61K 33/04; A61K 33/242; A61K 33/26; A61K 33/30; A61K 33/32; A61K 33/38; A61P 39/06

(57) ABSTRACT

Methods of treating or preventing an injury or related disease caused by ionizing irradiation exposure, including by photon, particle beam radiation, or both, in a subject in need thereof, the methods comprising administering to the subject a therapeutically effective amount of a surfactant, preferably an amphiphilic block copolymer or one or more of amphiphilic block copolymer polypeptide composites.

### CROSS-REFERENCE TO RELATED APPLICATIONS

This application claims the benefit of priority from the U.S. provisional patent application 63/617,495 filed Jan. 4, 2024, the entire disclosure of which is herein incorporated by reference in its entirety.

### TECHNICAL FIELD

The present invention relates generally to the areas of medicine that relate to the prevention or reduction of acute radiation sickness or delayed onset medical consequences in a subject resulting from exposure to a sufficient dose of any form of ionizing radiation.

### BACKGROUND

There is an increasing need for medical countermeasures that can therapeutically mitigate mammalian tissue, organ and total body injury resulting from exposure to toxic levels of ionizing irradiation.

Current events have highlighted various military or terrorist actions that establish a risk for a nuclear weapon detonation, or the dissemination of radioactive particles released from a radioactive material dispersal device (e.g., a dirty bomb). In such an event, population exposure to large doses of external and/or internal ionizing radiation is likely, in addition to traumatic injuries. In the event of exposure to large external doses of acute ionizing radiation Acute Radiation Syndrome (ARS) may result which could lead to death, long-term disability, or disease.

Additionally, a major risk associated with interstellar travel is exposure to intense ionizing irradiation in the form of sub-atomic particles such as electrons and nuclei as well as photon irradiation from various sources (Smart and Shea, 1985). A person exposed to such irradiation could also suffer disabling and/or fatal injury.

### SUMMARY

In an embodiment, the invention provides a method of treating an injury or related disease caused by ionizing irradiation exposure, including either photon or particle beam radiation, or both, in a subject in need thereof comprising administering to the subject a therapeutically effective amount of an amphiphilic block copolymer or amphiphilic block copolymer polypeptide composites.

In an embodiment, the invention provides a method of treating an injury or related disease caused by ionizing irradiation exposure, including either photon or particle beam radiation, or both, in a subject in need thereof comprising administering to the subject a therapeutically effective amount of an amphiphilic block copolymer compound or an amphiphilic block copolymer polypeptide compound or a composite of these compounds with cofactors such as antioxidants or other cell protective agents.

In an embodiment, the invention also provides compositions and methods of reducing ionizing radiation injury in a subject exposed to ionizing irradiation comprising administering to the subject a therapeutically effective amount of an amphiphilic block copolymer or amphiphilic block copolymer polypeptide compound or a composite of these compounds prior to the exposure to ionizing radiation.

In an embodiment, the invention further provides compositions and methods of preventing or treating the forms of acute radiation syndrome (ARS) which results from exposure to ionizing irradiation. ARS can take the form of Gastrointestinal Acute Radiation Syndrome (GI ARS), Neurological Acute Radiation Syndrome (NARS), or Hematological Acute Radiation Syndrome (HARS) in a subject. This embodiment applies to treatment of any combination of Acute Radiation Syndromes occurring separately or simultaneously to the subject.

In an embodiment, the invention additionally provides a method of promoting biological cell repair and oxygen free radical injury recovery comprising contacting the injured cells with an amphiphilic block copolymer. This embodiment applies to oxygen free radical injury resulting from ionizing radiation or secondary radical generation.

A therapeutic composition is comprised of a surfactant copolymer, a cofactor treatment consisting of an antioxidant, a cellular metabolic energy source, trehalose, a known radioprotectant, or any combination thereof.

To affect this goal, the barrier properties of damaged cellular membranes are reestablished by the surfactant copolymer, effectively “sealing” the injured membranes. The surfactant copolymer may also serve to reduce reactive oxygen intermediate (ROI) diffusion or propagation. To facilitate rapid tissue recovery, cellular energy levels can be reestablished through addition of a cellular metabolic energy source such as, for example, MgCl2-ATP which, serves a further dual benefit of improving the cellular ion balance. Addition of an antioxidant eliminates the generation or destructiveness of ROIs and enhances the safe metabolism.

### BRIEF DESCRIPTION OF THE DRAWINGS

These, as well as other objects and advantages of this invention, will be more completely understood and appreciated by referring to the following more detailed description of the presently preferred exemplary embodiments of the invention in conjunction with the accompanying drawings, of which:

FIG. 1 is an illustration of a representative process wherein ionizing radiation results in generation of oxygen free radicals in water or releases high energy subatomic particle beams that can act on the cell to cause structural disruption of the cellular membrane as well as damage to proteins and DNA.

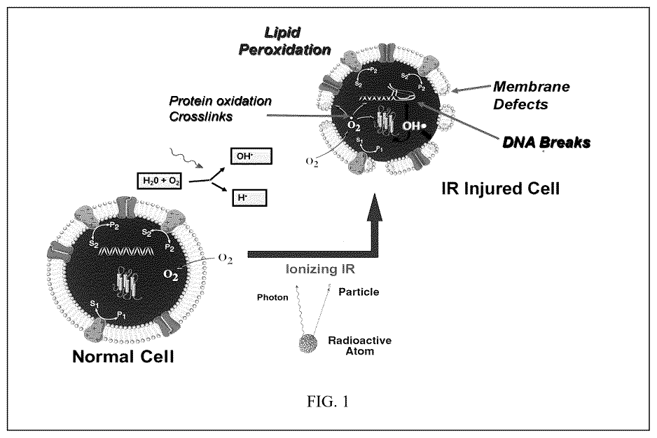

FIG. 2 is an illustration of the increased concentration of intracellular calcium in rat hindlimb skeletal muscle following intense X-Ray beam irradiation as compared to intracellular calcium concentration in rat hindlimb skeletal muscle that has not been exposed to irradiation. The increased incorporation of the calcium binding radiotracer Tc99m-pyrophosphate is indicative of the increase in concentration of intracellular calcium. In the animals exposed to X-ray irradiation, the comparative effectiveness of saline, Poloxamer 188 (P188), Polyethylene Glycol (PEG) intravenous injections on the intracellular calcium concentrations is displayed.

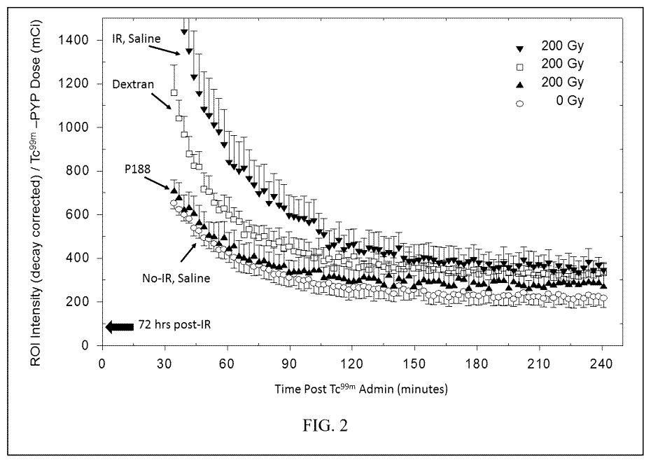

FIG. 3 is an illustration depicting chemical structures of representative amphiphilic block copolymers that may be utilized in this invention.

FIG. 4 is an illustration depicting the effects of a representative amphiphilic block copolymer-based surfactants applied to a cell membrane disrupted by ionizing irradiation.

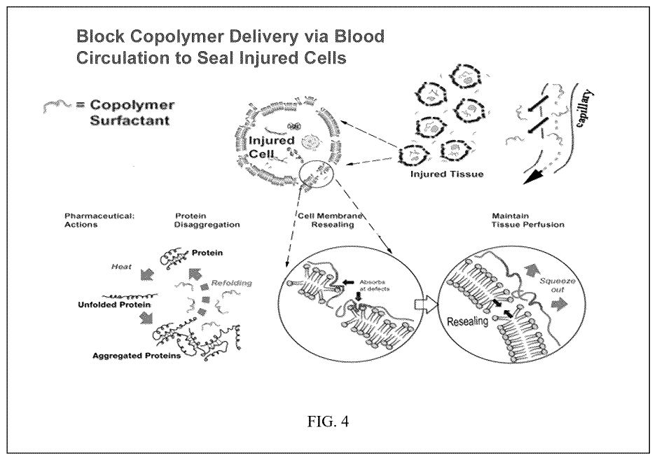

FIG. 5 is an illustration of comparative effectiveness of cell viability results for mammalian cells treated with different multiblock copolymers of this invention following exposure to 40 Gray (Gy) of radiation. Viability is determined by a fluorescence assay. Cells labelled with red fluorescence dye are not viable. Cells labelled with the green fluoresce dye are viable.

FIG. 6 is a plot of comparative viability of post-mitotic skeletal muscle cells maintained in standard cell culture medium at the end of a 4-hour test in which the different dishes of cells were either not irradiated (normal) or Co⁶⁰ gamma ray irradiated. The gamma irradiated cells were subjected to either 10 Gy, 40 Gy or 80 Gy irradiation at 21% oxygen saturation in the culture medium. The irradiated cells were either contacted with fresh cell culture medium or fresh cell culture medium containing I mM of an amphiphilic block copolymer known as Poloxamer 188 (P188) 30 minutes after the radiation exposure. The percentage cell viability in each dish of cells using the fluorescence viability test of FIG. 5. The increase in cell viability caused by contacting the cells with P188 is illustrated in these plots.

FIG. 7 is an illustration of gastrointestinal barrier function 6 days after effect of exposure to 12 Gy total body ionizing gamma irradiation when the animal is treated with highly purified free radical resistant P188 1 day after radiation exposure to prevent the occurrence of GI ARS which occurs due to ionizing irradiation doses of 6 Gy TBI or more.

FIG. 8 is an illustration of the 48 hr mean percent survival (±SEM) of non-IR sham-exposed adult rat skeletal muscle fiber cells, as well as survival of IR-exposed cells receiving P188 alone, PEG control, cofactor NAC alone as well as combined with P188 or MgATP and P188 with both MgATP and NAC.

### DETAILED DESCRIPTION

In all embodiments, the present invention provides a method of treating radiation sickness caused by exposure to ionizing radiation in a subject in need thereof via the administration of a therapeutically effective amount of a surfactant copolymer.

In all embodiments, the present invention can be used to protect mammalian animals from symptoms of ARS and death due to exposure to either photon or particle beam irradiation, or a combination of photon and particle beam.

In all embodiments, the present invention provides a method to restore molecular diffusion barrier function to mammalian cell membranes disrupted by ionizing radiation exposure. Without wishing to be bound by any theory, an amphiphilic block copolymer (e.g., a large molecular weight amphiphilic block copolymer) can restore functional and structural epithelial barrier function to organ systems and prevent trans-epithelial barrier transport of toxic molecules or micro-organisms into the blood circulation (Hannig and Lee, 2000; Terry et al., 1999).

The present inventors previously discovered that amphiphilic block copolymers administered to injured cells will concentrate at locations of exposed hydrophobic domains or damaged proteins or cell membranes in the body, such as a defect in a cell's plasma membrane lipid bilayer.

In all embodiments, to facilitate rapid tissue recovery, cellular energy levels can be re-established through addition of a cellular metabolic energy source which serves a dual benefit of improving the cellular ion balance and acting as an additional antioxidant. Thus, in all embodiments, the present invention can be comprised of a therapeutically effective amount of a surfactant copolymer, an antioxidant, a cellular metabolic energy source, or some combination thereof.

In all embodiments, the present invention decreases ROI generation and diffusion within the body following exposures to radiolytic decomposition of water (Codorniu-Hernandez and Kusalk, 2012; Bankura and Chandra, 2012).

An advantage of the present invention is that the therapeutic component acts to inhibit the cellular stress responses to misfolded or unfolded proteins that directly and indirectly result from toxic levels of ionizing irradiation exposure (U.S. Pat. No. 8,815,557).

The present inventors have proposed to combine therapeutic measures that prevent the generation of oxygen free radicals and enhance the capacity of tissues to metabolize ROI, thereby sealing damaged cell membranes and preventing further production of toxic oxygen radicals. Thus, in an illustrative embodiment, the invention provides methods and compositions for the treatment and prevention ARS and other injuries that might be caused or enhanced by the presence of oxygen free radicals.

In an embodiment, the surfactant copolymer is co-administered with an antioxidant to protect the surfactant copolymer from inactivation by the action of oxidative free radicals (Soneru et al., 2011).

In an embodiment, more than one surfactant copolymer, antioxidant, or cellular energy store may be combined in the invention. For example, it may be desirable to use a rapid release formulation of one cellular energy store agent in combination with an extended release formulation of the same or even a different cellular energy store agent. Thus, in an embodiment, the invention contains multiple surfactant copolymers, antioxidants, or cellular energy stores.

In an embodiment, the surfactant block copolymer is administered as a component of a radiation injury countermeasure that also contains other radiation countermeasures including trehalose, amifostine, N-acetyl cysteine amide, stem cells, tissue protective peptides, lypopeptides, purine or pyrimidine nucleosides with antioxidants, myeloid cells, anti-TNF antibodies, novel normuramyl glycopeptide compounds, antisense molecules, α,β-unsaturated aryl sulfones, benzimidazole, serpin molecules, fibroblast growth factors, peptides and analogs of heparin-binding growth factors, nicotinoyl ribosides and nicotinamide riboside derivatives, immune modulators, antiapoptotic agents, nitroxide agents, tri-substituted glycerol compounds, nanoparticle isoflavone compositions, recombinant flagellin, potassium channel inhibitors, methoxypolyethylene glycol chelate, or other countermeasures for ionizing radiation included in “Medical Countermeasures for unwanted CBRN exposures: part II radiological and nuclear threats with review of recent countermeasure patents” (Seed et al., 2016).

In an embodiment, one or more than one surfactant copolymer is administered to reduce mammalian cell and organ exposure to oxygen free radicals by slowing the transport of the oxygen free radicals in and around the cells or in and around the organs. Without wishing to be bound by any theory, the presence of the copolymer reduces the diffusivity of the water, thereby limiting the distance traveled by the free radicals.

In an embodiment, one or more than one surfactant copolymer is administered as a radioprotectant prior to radiation exposure to reduce the diffusivity of water, thereby reducing the toxicity of free radicals that are generated in response to exposure to ionizing irradiation, enabling greater capture of free radicals by antioxidants.

### Ionizing Radiation Damage

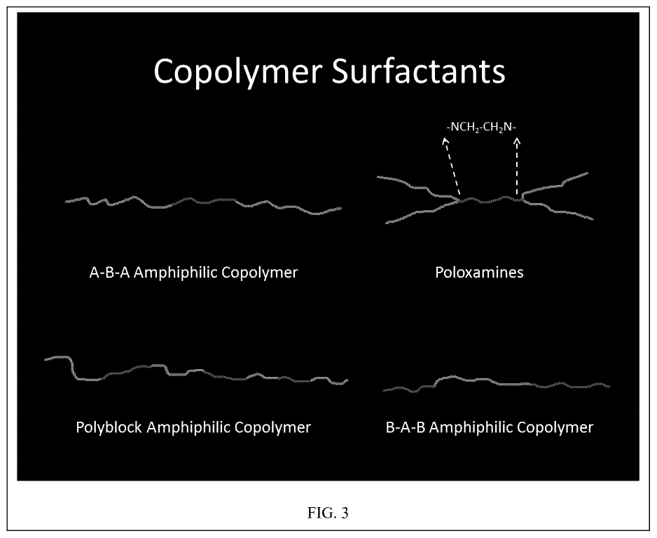

The two primary mechanisms of radiation alteration or damage to biological macromolecules are direct structural alteration by subatomic particle beam irradiation or indirect alteration by photon ionizing irradiation generation of oxygen-based radicals in water. In the latter mechanism, energy from the incident radiation waves excites water molecules, breaking the bonds that hold the molecules together and producing hydrogen and hydroxyl radicals (Citrin and Mitchell, 2017). These compounds, often referred to as ROIs, can combine to form toxic hydrogen peroxide or cause further damage to the cell. Hydroxyl radicals are particularly reactive and can cause damage to many biomolecules via oxidation, including the lipids that comprise cell membranes to cause structural damage as illustrated in FIG. 3 (Hannig and Lee 2000). Furthermore, ROI diffusion from irradiated cells is a widely suspected cause of the radiation-induced bystander effects, by which non-irradiated cells are damaged due to proximity to irradiated cells (Desouky et al., 2015).

Hydroxyl radicals can also trigger cascading reactions to generate further harmful ROIs such as peroxyl and superoxyl radicals. For example, the reaction of a single hydroxyl radical can form as many as 34 harmful peroxides (Chaplin, 2022). In the presence of oxygen, lipid peroxidation also occurs. In the mechanism by which this occurs, a hydroxyl radical pulls off a hydrogen atom from a nearby functional group on tissue, becoming water and forming a radical on the tissue. Then an oxygen diradical (singlet oxygen) reacts with the radical to form a peroxide radical which can pull off a hydrogen atom to form hydroperoxide. At a lipid bilayer, hydroxyl and peroxide radicals can interact with a reactive methylene hydrogen atom to form a fatty acid radical, which can generate a lipid peroxidation chain reaction to degrade the membrane until two lipid radicals react to form a non-radical and break the chain (Kiang et al., 2011).

### Medical Consequences Of Ionizing Radiation Damage

Acute Radiation Syndrome (ARS) is the resulting injury and illness following exposure to high doses of external, penetrating ionizing radiation to a significant portion of the body (CDC). The most likely cause of ARS is exposure to a nuclear detonation or radiation disaster, such as the Chernobyl Nuclear Power Plant event. Though rare, the impact of these events would be disastrous given the severity of and difficulty of treating ARS. Risk of ARS is a growing concern as global tensions and the threat of nuclear warfare increases.

While the severity of symptoms following radiation exposure is dose-dependent, the mechanism by which radiation inflicts cellular damage remains the same. Thus, in some embodiments the methods described herein are applicable to the symptoms of all forms of ARS which can be GIARS, HARS, cardiovascular radiation syndrome (CV RS), and ANS. Such radiation syndromes can also be caused by toxic levels of any form of ionizing irradiation exposure.

In some embodiments the radiation can take the form of, for example, X-rays, gamma rays, ultraviolet rays, alpha particles, beta particles, or neutron particles. The damage can be inflicted by any radiation emitting source. In some embodiments the source is, for example, radiation therapy, a nuclear detonation, cosmic background radiation, or galactic radiation.

Currently, the United States FDA-approved medical treatments ARS are few in number and limited in function. Three of these treatments are growth factors and cytokines which function primarily by promoting and supporting neutrophilic function and stimulating myeloid progenitors in the bone marrow. A fourth stimulates and restores blood platelet levels to mitigate hemorrhaging after radiation exposure. GIARS currently has no US FDA-approved treatment (Singh and Seed, 2020).

The short term death of cells exposed to high doses of radiation is known to be caused by the permeabilization of cellular membranes and subsequent loss of molecular transport barrier function, particularly to salt ions (Hannig and Lee, 2000). Results from prior studies indicate that cells contacted with block copolymer surfactants are rescued from short term cellular death following ionizing irradiation exposure. This prevents massive ATP loss and cell death. Our results strongly suggest that the efficacy of P188 treatments can be enhanced with the addition of an antioxidant and cellular energy source cofactor as is considered for this invention. Addition of these three compounds to irradiated cells results in an 18 hour viability that is nearly commensurate with cells that received no radiation treatment.

### Copolymers As Radiation Countermeasures Or Radioprotectants

Amphiphilic block copolymers serve as active agents in the invented methods. Amphiphilic block copolymers are desirable for multiple reasons as they have low detergency, high biocompatibility, do not denature proteins, and do not disrupt cell membranes. Additionally, amphiphilic block copolymers are easily synthesized, have highly tunable molecular weights, and many are approved by the Food and Drug Administration (FDA). Without wishing to be bound by theory, it is believed that the hydrophilic blocks are able to disrupt the water structure surrounding the disrupted cell membranes and misfolded proteins, while the hydrophobic blocks bind to exposed hydrophobic domains of irradiated membranes and misfolded proteins to catalyze the sealing of disrupted membranes or disaggregate misfolded proteins or both. Regardless of mechanism, amphiphilic block copolymers have exhibited properties that are useful in mitigating the symptoms of ARS, including the ability to seal permeabilized cell membranes and reduce biomolecular leakage from cells (Lee, 2002; Terry et al., 1999; Yasuda et al., 2005).

In one common embodiment, the sequential addition of two or more alkylene oxides to a low molecular weight water soluble organic compound containing one or more active hydrogen atoms. These latter compounds are described in U.S. Pat. No. 5,470,568, which is herein incorporated as a reference.

The amphiphilic block copolymer comprises both hydrophilic (polar) (“A”) and hydrophobic (nonpolar) (“B”) regions and acts as a surfactant. The block copolymer structure can be hydrophilic-hydrophobic-hydrophilic (ABA), hydrophobic-hydrophilic-hydrophobic (BAB), or have a core structure (A or B) with two or more pendant side chains of the structure-A (if a B core), -B (if an A core), -AB, -BA, -ABA, -BAB, or a combination thereof. An amphiphilic block copolymer that is biocompatible and/or FDA approved is preferred.

In general, the amphiphilic block copolymer comprises at least one (e.g., 1, 2, 3, 4, 5, etc.) hydrophobic block and at least one (e.g., 1, 2, 3, 4, 5, etc.) hydrophilic block. The hydrophilicity and hydrophobicity can be measured, if necessary, by any suitable method. For example, the water contact angle (0), which measures the wettability of a material, of a comonomer, homopolymer segment, prepolymer, and/or copolymer can be measured using, e.g., a goniometer. A material with a contact angle (θ) of greater than 90° is considered to be hydrophobic, and a contact angle (θ) of less than 90° is considered to be hydrophilic.

In some embodiments, the hydrophobic block comprises repeat units selected from a hydrophobic polypeptide, polyoxypropylene, polystyrene, polyglycolide, polylactide, poly(lactic-glycoacid), polycaprolactone, hydrophobic polyurethane, polyester, poly-N-isopropylacrylamide, polymethylmethacrylate, poly(2-dimethylaminoethylmethacrylate), polyethylene, polypropylene, polyisoprene, polybutylene, polybutadiene, poly(styrene-butadiene), polyvinyl chloride, polytetrafluoroethylene, polydimethylsiloxane, and a combination thereof. In some embodiments, the hydrophilic block comprises repeat units selected from polyethylene oxide, polyvinyl alcohol, hydrophilic polyurethane, polyvinylpyrrolidone, polyacrylamide, polyacrylic acid, poly(meth)acrylic acid, polyethylenimine, poly(methyl vinyl ether), poly(styrene-maleic anhydride), polyethylene glycol ether, polyamine, a hydrophilic polypeptide, and a combination thereof.

In some embodiments, the hydrophilic block comprises repeat units selected from polyethylene oxide, polyvinyl alcohol, hydrophilic polyurethane, polyvinylpyrrolidone, polyacrylamide, polyacrylic acid, poly(meth)acrylic acid, polyethylenimine, poly(methyl vinyl ether), poly(styrene-maleic anhydride), polyethylene glycol ether, polyamine, a hydrophilic polypeptide, and a combination thereof.

Polyurethanes are prepared from polyols and diisocyanates to form repeat units with an—NH—CO-O-linkage. Whether a polyurethane is hydrophilic or hydrophobic typically depends on monomers used. In general, the hydrophilic to hydrophobic content ratio can be controlled by using a mixture of polyols with varying hydrophilicities and/or the use of chain extenders. The polyols are generally based on polyesters, polyethers, mixtures thereof, and copolymers of esters with ethers. Polyurethanes based on polyethylene oxide are highly hydrophilic materials.

Suitable examples of diisocyanates include 1,6-hexamethylene diisocyanate, 1,4-diisocyanato butane, L-lysine diisocyanate, isophorone diisocyanate, 1,4-diisocyanato 2-methyl butane, 2,3-diisocyanato 2,3-dimethyl butane, 1,4-di (1 propoxy-3-diisocyanate, 1,4-diisocyanato 2-butene, 1,10-diisocyanato decane, ethylene diisocyanate, 2,5 bis(2-isocyanato ethyl) furan, 1,6-diisocyanato 2,5-diethyl hexane, 1,6-diisocyanato 3-methoxy hexane, 1,5 diisocyanato pentane, 1,12-dodecamethylene diisocyanate, 2 methyl-2,4 diisocyanato pentane, 2,2 dimethyl-1,5 diisocyanato pentane, ethyl phosphonyl diisocyanate, 2,2,4-trimethyl-1,6-hexamethylene diisocyanate, 4,4′-diphenylmethane diisocyanate, 2,4′-diphenylmethane diisocyanate and 2,2′-diphenylmethane diisocyanate; mixtures of 2,4′-diphenylmethane diisocyanate and 4,4′-diphenylmethane diisocyanate, 2,4-toluene diisocyanate, mixtures of 2,4-toluene diisocyanate and 2,6-toluene diisocyanate, 2,4′-diphenylmethane diisocyanates, 4,4′-1-diphenylethane diisocyanato, 1,5-naphthylene diisocyanate, and combinations thereof.

The chain extenders are low molecular weight diols, diamines, triols, triamines or higher molecular weight oligomeric units having the functionality of two or higher. Suitable chain extenders include water, aliphatic difunctional or trifunctional alcohols, amines, aminoalcohols, aminoacids, and hydroxyacids. Specific examples include 2-aminoethanol, 2-dibutylaminoethanol, n-alkyldiethanolamines, n-methyl-diethanolamine, ethylene diol, diethylene diol, 1,4-butanediol, propylene diol, dipropylene diol, 1,6-hexanediol, isosorbide (1,4:3,6-dianhydrosorbitol), glycerol, ethylene diamine, tetramethylene diamine, hexamethylene diamine, isophorone diamine, propanolamine, ethanolamine, glycyl-L-glutamine, glycyl-L-tyrosine, L-glutathione, glycylglycine, L-malic acid, and combinations thereof.

In certain preferred embodiments, the amphiphilic block copolymer comprises repeat units comprising a polypeptide, a poloxamer, a meroxapol, a poloxamine, a polyol, a polyethylenimine, a styrene maleic anhydride, or a combination thereof in di-block, tri-block, tetra-block or more compositions.

A polypeptide can be hydrophilic or hydrophobic depending on the particular amino acids forming the polypeptide. Hydrophobic amino acids include glycine, alanine, valine, leucine, isoleucine, proline, phenylalanine, methionine, and tryptophan. Hydrophilic amino acids have side chains that are polar but not charged, including serine, threonine, cysteine, asparagine, glutamine, and tyrosine. The polypeptide can include any desirable sequence of two or more amino acids to provide the desired hydrophilicity or hydrophobicity.

In some embodiments the contemplated amphiphilic copolymers include polysaccharides, polyglyercol, saccharide, the poloxamers, the meroxapols, the poloxamines, polypeptides, and the PLURADOT.™. polyols or some combination thereof.

In some embodiments, the contemplated amphiphilic copolymer is poloxamer 188, which is already FDA-approved in the synthetic blood replacement Fluosol (Check and Hunter, 1988, U.S. Pat. Nos. 4,879,109; 4,897,263; 4,937,070, incorporated herein by reference).

In some embodiments, the contemplated amphiphilic copolymer is poloxamine 1107, which has been shown to be effective in reducing cellular necrosis and biomolecule leakage from cells due to radiation injury (Hannig et al., 1998).

In some embodiments, the active surfactant comprises block copolymers that are available from BASF Corp.

In some embodiments, the molecular polydispersity index has been reduced by “purifying” the copolymer which effectively removes lower and higher molecular weight copolymers or copolymer fragments from the embodied copolymer.

In some embodiments, the peroxide content of the copolymer has been limited by “purifying” the copolymer which protects the polymer from generation of degradants.

a. Surfactant Copolymer Molecular Weight Specifications

In some embodiments, the amphiphilic block copolymer can have an average molecular weight within the range of 1,000 to 30,000 g/mol, where 1 g/mol is defined as 1 Dalton.

In one embodiment of the invention, the use of a poloxamer with a molecular weight of 2,000 to 20,000 Daltons is considered. This range is highly soluble. In some embodiments the hydrophobic group of the poloxamer constitutes 45-95% of the molecular weight and the hydrophilic groups constitute 5-55% of the molecular weight.

The phrase “large molecular weight,” as used herein refers to a copolymer with a molecular weight of about 8,000 g/mol or more. The phrase “small molecular weight,” as used herein refers to a copolymer with a molecular weight of less than about 8,000 g/mol.

In some embodiments, the number average molecular weight can be measured by any suitable method, including gel permeation chromatography (GPC) or size exclusion chromatography (SEC). In a preferred embodiment, the number average molecular weight is measured by GPC.

b. Surfactant Water Solubility Properties

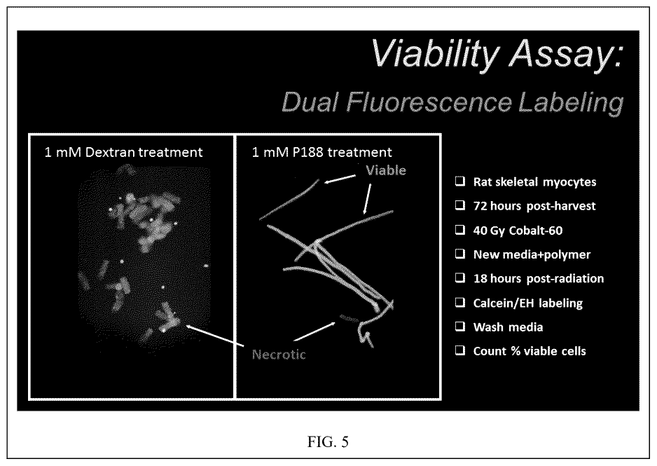

Surfactant copolymers are capable of preventing or minimizing cell membrane permeabilization and repairing damaged membranes as illustrated in FIG. 5 and as described in U.S. Pat. No. 5,605,687 and U.S. Patent Pubs. US2003/0118545A1 and US2005/0069520A1, which are herein incorporated by reference. While integrin receptors are typically independently responsible for cell adhesion to matrix components, studies of various block copolymers indicate that localization between surfactant chaperones and the cell membrane is a result of favorable interactions between hydrophobic domains. It has been shown that the polymer inserts into lipid films as a function of lipid packing density and the hydrophobic central domain of the polymer binds to the internal hydrophobic portion of the lipid bilayer when those groups are exposed (Maskarinec and Lee, 2003). Such an exposure occurs following a damaging event, such as radiation subjection or trauma resulting in an open wound. The manner in which the poloxamer is folded when this binding occurs has been postulated to assist in the restoration of a non-adhesive cell surface. Poloxamers are surprisingly capable not merely of restoring a non-adhesive surface, but actually of repairing or facilitating the repair of complete permeations of the membrane bilayer.

c. Radiation Resistant Copolymers

In one or more embodiments of this invention, the administered polymers of block copolymers can be damaged by reactive oxygen species produced by ionizing irradiation or radiation injury related cellular stress. To maximize the effectiveness of polymers and copolymers as radiation protectants or countermeasures, the polymers and amphiphilic block copolymers will be formulated with minimal hydroperoxide, carbonyl, aldehyde and other chemical side groups on the polymers that increase the reactivity of the copolymer with reactive oxygen species.

d. Cofactors

In some embodiments, it is considered that an antioxidant will be administered as a cofactor of the countermeasure to free radicals generated by radiation injury. It has been found that antioxidants are capable of stabilizing block copolymers, as well as protecting against symptoms associated with ARS in the intestine (Parks et al., 1982; Granger at al., 1986; Nalini et al., 1993), central nervous system (Hall et al., 1988), heart (Levitt et al., 1994), lung (Aeba et al., 1992), liver (Cosenza et al., 1994) and kidney (Shackleton et al., 1994). A wide variety of antioxidants are contemplated as being useful for the treatment of free radical mediated injury of the cellular membrane by acting as free radical scavengers and inhibitors of lipid peroxidation. Antioxidants have been studied extensively in these capacities as therapy for reperfusion injuries with mixed results during clinical trials (Becker, 2004). For example, lazaroids have produced inconsistent results in cases of intestinal protection, with some studies finding reduction of mucosal injury (Stone et al., 1992; Katz et al., 1995) and some studies finding no benefit of lazaroid administration (Park et al., 1994; Van Ye et al., 1993. Superoxide dismutase (SOD) has similarly shown promise in some studies (Flaherty, 1991; Land et al., 1994), and no significant benefit in others (Vanhaecke, 1991; Euler, 1995; Pollac et al., 1993; Flaherty et al., 1994) in animal models of reperfusion injury. Allopurinol has also been found to reduce lipid peroxidation in tissue post-surgery (Coghlan et al., 1994; Rashid and William-Olsson, 1991). One or more antioxidants may be used in combination with each other along with a suitable surfactant copolymer and a cellular energy store. Compositions having antioxidant properties and contemplated as being useful in the invention have been previously described in U.S. Pat. Nos. 5,725,839; 5,696,109; 5,691,360; 5,683,982; 5,659,055; 5,659,049; 5,648,377; 5,646,149; 5,643,943 and 5,623,052, all of which are incorporated herein by reference.

Illustrative compositions having antioxidant properties which are contemplated as being useful in the invention include ascorbic acid (ascorbate or Vitamin C), tocopherol (vitamin E), Vitamin A, mannitol, .beta.-carotene, bioflavonoids, flavonoids, flavones, flavonols, proanthocyanidins, selenium, glutathione, N-acetyl cysteine (NAC), superoxide dismutase (SOD), lipoic acid, and coenzyme Q-10 (CoQ10), lazaroids, allopurinol, carotenoids such as lycoprene, lutein and polyphenols, and hindered phenols such as BHT and BHA. Transition metals such as Ag, Au, and Zn are also contemplated for this purpose. In some embodiments, transition metal complexes are contemplated for their abilities as radical oxygen scavengers. Such contemplations include Mn and Fe complexes of ligands, small molecular weight compounds such as pentaazamacrocycles, Salen derivatives, and porphyrins, and iron and copper chelate complexes (Timoshnikov et al., 2022). While ionized metals can be strong oxidants and cause polyether damage, complexes between transition metals and natural compounds, particularly ligands, have been investigated for their ability to act as antioxidants, prooxidants, and free radical scavengers (Kostova and Balkansky, 2013, Choudhary et al., 2011). Another approach would be to complex the antioxidant to the surfactant copolymer via hydrophobic attraction or chemical interaction which will simply deliver the agent to the site of damage.

It is understood that certain antioxidants may be more desirable for use before vs after the ROI instigating event, e.g. radiation exposure. It is also understood that certain antioxidants when combined may have a greater than additive effect.

### a. Example: Lipoic Acid

Lipoic acid is specifically contemplated as an antioxidant as it is considered a universal antioxidant, capable of free radical scavenging directly by reacting with ROIs or indirectly by promoting antioxidant enzymes. As a result, it is one of the foremost antioxidants considered for this invention. The PEGylation of lipoic acid is proposed to occur via esterification between the carboxylic group of the lipoic acid and the hydroxyl end of the PEG molecule. As a result, the contemplated surfactant copolymers can be functionalized with a lipoic acid antioxidant. By heating the solution, lipoic acid can also be polymerized further increasing the amount of antioxidant that might be used with a given low surfactant copolymer concentration (Han et al, 2022, Yang et al, 2018).

### b. Example: Trehalose

Trehalose is a disaccharide that is known for its radioprotection properties. This is in part due to its ability to absorb onto proteins and nucleic acids in a way that decreases water activity (Weng et al., 2016). This way trehalose acts as a cytoprotectant and as an ionizing radiation protectant (Yoshinaga et al., 1997; Liu et al., 2017). Similarly, trehalose has been shown to influence the water phase behavior of P188 (Tale et al., 2014). Furthermore, trehalose is an antioxidant that captures free electrons (Matros et al., 2015).

In one embodiment of the compositions claimed in this patent, trehalose will be used to protect both irradiated cells and the amphiphilic block copolymer surfactants from ionizing irradiation damage.

Trehalose will also be co-administered with the surfactant copolymer when it is administered after ionizing irradiation exposure to protect the copolymer from oxidative damage caused by reactive oxygen species released from the cells and tissues injured by the ionizing irradiation.

c. Metabolic Energy Charger

In instances of radiation exposure, when the cellular membrane is damaged, the normal barrier function of the cell membrane is lost, and cell energy is greatly depleted in an effort to maintain ion gradients inside and outside the cell. As the cell energy is depleted, levels of calcium and other ions rise in the cell, which can lead to further formation of damaging free radicals and cell death signals. As such, in some embodiments, the co-administration of a cellular energy source is also contemplated.

The most common form of stored cellular energy is high energy phosphate compounds. High energy phosphate compounds generally comprise pyrophosphate bonds and acid anhydride linkages formed by taking phosphoric acid derivatives and dehydrating them. High energy phosphate compounds react in a variety of cellular processes to provide energy to the cell, controlling the process by coupling with a particular nucleoside and driving the process to favor the forward reaction.

ATP is the high energy phosphate compound found generally in all cells. ATP comprises an ordered carbon backbone having a triphosphate. Removing one of the phosphate groups from ATP releases stored energy for use within the various cellular processes and consequently results in the formation of Adenosine Diphosphate (ADP). ADP can be subsequently converted back to ATP through the oxidation of glucose in the Krebs cycle such that stored energy in the form of ATP is again available to the cell.

In some embodiments of the methods described herein, the method further comprises administering a therapeutically effective amount of a cellular energy source as a cofactor. In some embodiments, such a cellular energy source is selected from a group comprising ATP, ADP, MgCl.sub.2-ATP, creatine-phosphate, or some combination thereof.

### Administration and Dosage

a. Carriers

The methods described herein comprise using (e.g., administering) a surfactant copolymer in the form of a pharmaceutical composition. In particular, a pharmaceutical composition will comprise at least one surfactant copolymer, as described herein, and a pharmaceutically acceptable carrier. The pharmaceutically acceptable excipients described herein, for example, vehicles, adjuvants, carriers or diluents, are well-known to those who are skilled in the art and are readily available to the public. Typically, the pharmaceutically acceptable carrier is one that is chemically inert to the surfactant copolymer and has no detrimental side effects or toxicity under the conditions of use.

b. Administration Form and Route

In some embodiments, aqueous compositions of the present invention comprise an effective amount of the previously defined surfactant copolymers and cofactor treatment dissolved or dispersed in a pharmaceutically acceptable carrier or aqueous medium. The phrases “pharmaceutically or pharmacologically acceptable” refer to molecular entities and compositions that meet the United States FDA Office of Biologics standards.

As used herein, “pharmaceutically acceptable carrier” includes any and all solvents dispersion media, coatings, antibacterial and antifungal agents, isotonic and absorption delaying agents, pharmaceutical preservatives, oils and suitable mixtures thereof. The use of such media and agents for pharmaceutical active substances is well-established. Except insofar as any conventional media or agent is incompatible with the active ingredient, its use in the therapeutic compositions is contemplated as a component of this invention. Supplementary active ingredients can also be incorporated into the compositions. For human administration, pharmaceutical preparations are expected to meet sterility, pyrogenicity, and general safety and purity standards as required by the FDA Office of Biologics standards.

The biological material should be extensively purified by standard pharmaceutical separation techniques to remove undesired polymer fragments or other small molecular weight molecules and/or lyophilized for more ready formulation into a desired vehicle, where appropriate.

In some embodiments, the pharmaceutical composition can be administered as oral, sublingual, transdermal, subcutaneous, topical, absorption through epithelial or mucocutaneous linings, intravenous, intranasal, intraarterial, intramuscular, inter-peritoneal, intrathecal, rectal, vaginal, or aerosol formulations.

In some embodiments, formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, preservatives, and oils.

The oral carriers may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening or flavoring agent, such as sucrose, lactose or saccharin. When the dosage unit form is a capsule it may contain, in addition to compositions of the above type, a liquid carrier.

Various other compositions may be present as coatings or to otherwise modify the physical form of the dosage unit. For instance, tablets, pills or capsules may be coated with shellac, sugar or both. A syrup or elixir may contain the nonactive compounds sucrose as a sweetening agent, methyl and propylparabens as preservatives, a dye, and flavoring such as cherry or orange flavor.

In other embodiments, the composition may be administered via liposomal formulations or creams. In some embodiments, such compositions can be prepared as sterile injectables, either as liquid solutions or suspensions; solid forms suitable for using to prepare solutions or suspensions upon the addition of a liquid prior to injection can also be prepared; and the preparations can also be emulsified. In some embodiments, the injectable carrier includes sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid to the extent that it can be easily injected via a syringe. It must be stable under the conditions of manufacture and storage and must be preserved against the contaminating action of microorganisms, such as bacteria and fungi.

c. Dosage

Individual recipients of toxic levels of ionizing irradiation may respond better to one combination of drugs in one set of circumstances and in another set of circumstances respond more favorably to a different drug combination. Further, certain combinations of drugs are recommended only for certain conditions and some drugs may not be effectively combined. Additional factors that are well known to influence patient response to drug therapy include, but are not limited to, species, age, weight, gender, health, pregnancy, addictions, allergies, ethnic origin, prior medical conditions, current medical condition and length of treatment. Thus, it is recognized that in the practice of this invention a wide variety of dosages may be useful and the administrator will be well acquainted with the need to individualize dosage(s) and the route(s) of administration to each patient.

Both the route and form of administration can significantly influence the dosage. For example, the dosage used with the oral administration of a drug in an extended release form may be more than ten-fold greater than the dosage of the same drug administered intravenously. Thus in preferred embodiments, the therapeutic dosage is the deemed “effective amount” of surfactant copolymer, antioxidant, and metabolic energy source. Various general considerations taken into account in determining the “effective amount” are known to those of skill in the art and are described, e.g., in Gilman et al., eds., Goodman And Gilman's: The Pharmacological Bases of Therapeutics, 13th ed., Pergamon Press, 2017, which is herein incorporated by reference.

For example, a therapeutic composition of the present invention could comprise a therapeutically effective dose of surfactant copolymer, such as, for example, a poloxamer, a meroxapol, a poloxamines, a PLURADOT®. polyols and combinations thereof in an amount ranging from 0.01 mg/ml of blood volume to 20.0 mg/ml blood volume or 1.0% to 10.0% w/v. Additional ranges of surfactant copolymer dosages are contemplated.

An “effective amount” means an amount sufficient to show a meaningful benefit in an individual, organ, or cell to be treated. A meaningful benefit includes, for example, detectably treating, relieving, or lessening one or more symptoms of an illness caused by radiation injury (e.g., nausea, vomiting, headache, diarrhea, fatigue, seizures, skin damage, gut permeability, organ failure, pain), inhibiting, arresting development, preventing, or halting further development of the illness, reducing the incidence of ARS, preventing death of irradiated tissue, promoting cell repair and recovery to increase survival of irradiated cells, and/or inhibition of an unfolded protein response in irradiated tissue. The meaningful benefit observed in the subject, cell, or tissue to be treated can be to any suitable degree (10, 20, 30, 40, 50, 60, 70, 80, 90% or more). In some aspects, one or more symptoms of the disease are prevented, reduced, halted, or eliminated subsequent to administration of the composition described herein, thereby effectively treating the illness to at least some degree.

Representative therapeutically effective dose ranges of antioxidant cofactors can comprise: TABLE-US-00001 Antioxidant Dose Level (mg) Vitamin C 100-10,000; CoQ10 5-50; NAC 25-1,000, or ALA 250-5000.

Representative therapeutic compositions further include cellular energy sources, such as, for example, Adenosine Triphosphate (ATP), Adenosine Diphosphate (ADP) and phosphocreatine, at therapeutically effective dose levels from about 0.1% to about 1.5% w/v (component weight to volume of composition).

In some representative embodiments, therapeutic treatments can comprise dual administration of a therapeutic composition such as, for example, combined administration of two or more suitable intravenous, intramuscular, or topical compositions.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be achieved. All such similar substitutes and modifications apparent to those skilled in the art are deemed to be within the spirit, scope and concept of the invention as defined by the appended claims.

### Examples

The present inventors have conducted several experiments to illustrate the benefits of therapeutic composition of the present invention.

i. Viability Testing in Flexor Digitorum Brevis Skeletal Muscle Cells

In order to illustrate the therapeutic capability of the present invention as a medical countermeasure against gamma irradiation, mammalian rat cells were harvested, exposed to directly ionizing radiation (IR) resulting in damage to cellular membranes, proteins and DNA, and treated with therapeutic groups including surfactant copolymer P188, antioxidant n-acetyl-cysteine (NAC), cellular energy store MgCl₂-ATP, and combinations thereof. Notably, similar peroxidation of the cellular membrane is achieved through exposure to a variety of alternative systemic or local tissue injuries such as, for example, electrical shock, acute myocardial infarction, burn trauma, stroke, spinal cord injury, head injury, organ transplantation, and conditions characterized by exposure to chemical oxidants.

a. Compositions and Methods

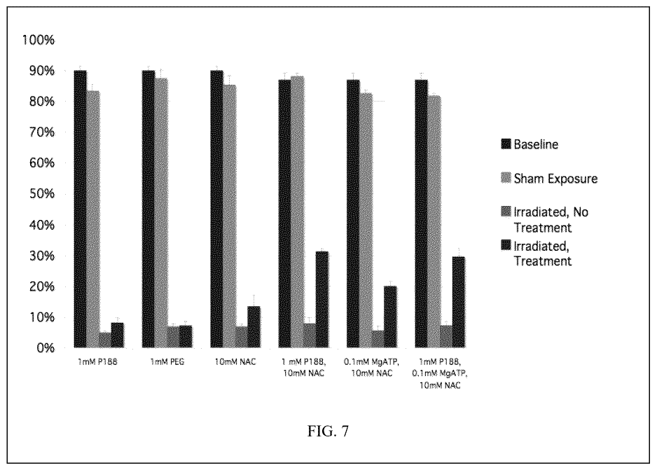

Intact rat adult skeletal muscle fibers were harvested from Spague-Dawley rats were divided into 8 treatment conditions to evaluate the efficacy of the invention in preserving muscle fiber viability against 40 Gy radiation exposures. The treatment groups are indicated in FIG. 7.

Following irradiation (or sham exposure) of muscle fiber batches all the cell batches were returned to the tissue culture lab where various treatments were added to the irradiated dishes to determine the effects of Dextran treatment versus P188 treatment, and 48 hour cell viability data. Sham-exposed dishes as well as the remaining IR exposed dishes received additional media culture equivalent to the amount added to the dishes receiving the various therapeutic treatments.

Fluorescent dye was added at 18 and 48 hours post IR exposure to the cell batches in order to observe survival in the same manner used for initial viability testing. The viability of cells at 48 hours of testing is illustrated in FIG. 7 and were determined as the percentage of cells exhibiting calcine fluorescence alone. Our analysis considered the mean percentage viability for the multiple samples done for each testing parameter at 18 hours post-IR exposure.

b. Results

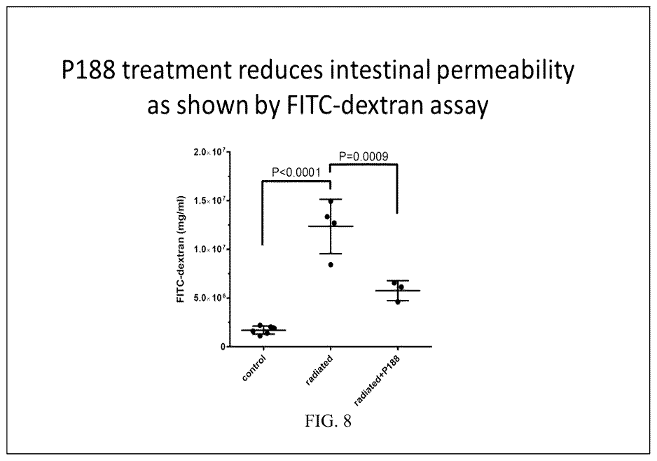

As illustrated in FIG. 8, the relative radiation countermeasure efficacy of P188 was compared to 10 kDa polyethylene glycol (PEG) for polymer treatment of IR exposed skeletal muscle fibers. While treatment of 1 mM PEG offered a no significant improvement versus sham treatment of IR exposed cells with respect to cell viability, treatment of IR exposed cells with 1 mM of P188 offered significantly better efficacy.

Using the same experimental methodologies as above, we examined the viability of cofactor-treated cells with and without addition of P188 at 48 hours following irradiation. Cells that received both cofactor and P188 demonstrated statistically significant improved survival (29.0%. +−. 2.3) versus irradiated cells receiving no treatment (8.6%. +−. 2.1). Irradiated cells treated with cofactor alone also showed an increased survival versus those receiving no treatment (19.9% vs. 2.9%). Additionally, the group treated with combination of cofactors and P188 had manifested better survival than those treated with cofactors alone (p<0.05).

ii. Cellular Membrane Repair and Viability After High Dose Irradiation

Further evaluation of the impact of surfactant copolymers such as P188 on cell viability, specifically due to membrane function, after radiation exposure was conducted via fluorescence imaging.

a. Compositions and Methods

Rat skeletal myocytes were harvested and grown in cell cultures labelled with Calcein-AM+EtH. 72 hours after harvesting, cultures were treated with 40 Gy Cobalt-60 radiation. Cultures were treated with either 1 mM 10 kDa neutral Dextran or 1 mM P188 and imaged. Cultures were also treated with 10, 40, and 80 Gy doses to evaluate the efficacy of P188 across a range of radiation doses compared to untreated cells.

b. Results

Representative fluorescence results are indicated in FIG. 5. Due to Calcein-AM+EtH labelling, red fluorescence indicates necrotic cells which have lost membrane integrity and green fluorescence indicates viable cells with intact membranes. The representative image shown indicates the dramatic increase in cell viability as a result of treatment with P188.

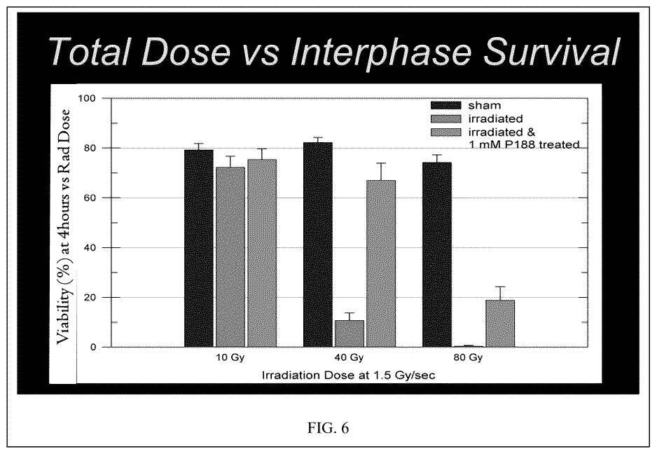

Radiation dose response results are illustrated in FIG. 6. At 10 Gy, the difference between sham and irradiated cells was insignificant. However, at higher radiation doses, P188 displayed a dramatic increase in cell viability compared to untreated cells (from approximately 10% to 65% and 0% to 20% for 40 and 80 Gy respectively).

iii. P188 Radiation Dose Response

P188 has also been evaluated at a range of radiation doses in order to determine the impact of radiation dose on cell viability compared to untreated cells.

a. Compositions and Methods

Rat skeletal myocytes were harvested and grown in cell cultures labelled with Calcein-AM+EtH. 72 hours after harvesting, cultures were treated with 40 Gy Cobalt-60 radiation. Cultures were treated with either 1 mM 10 kDa neutral Dextran or 1 mM P188 and imaged.

b. Results

Representative fluorescence results are indicated in FIG. 5. Due to Calcein-AM+EtH labelling, red fluorescence indicates necrotic cells which have lost membrane integrity and green fluorescence indicates viable cells with intact membranes. The representative image shown indicates the dramatic increase in cell viability as a result of treatment with P188.

iv. Gut Permeability in Mice After LD50 Exposure

In order to illustrate the efficacy of the present invention in the treatment of GI ARS symptoms, the effect of P188 on gut permeability.

a. Compositions and Methods

C57BL/6 mice received total body gamma irradiation (Cs¹²⁷ source) doses of 14 Gy on Day 0. The control group was mock radiated without exposing the core. One day post-irradiation (24 hours), mice were injected subcutaneously beneath the back skin with either saline (sham) or purified P188 (1250 mg/kg). On Days 2 and 3 post-irradiation, all mice were given 0.5 ml saline solution intraperitoneally to improve hydration and ensure sufficient serum volume at the time of collection.

Health of animals was monitored for either 4 or 6 days using single blind Nunamaker's scoring. After 12 hours of fasting on day 4 or 6 post irradiation, mice were orally gavaged with FITC-dextran. Blood was collected via cardiac puncture 4 h post FITC-dextran gavage and intestines collected for histology in 10% NBF.

b. Results

As illustrated in FIG. 8, treatment with P188 produced a statistically significant decrease in FITC-dextran release from the gut following radiation exposure (p<0.005). This result indicates a restoration of gastrointestinal mucosal barrier function, a primary symptom of GI ARS.

### CITED REFERENCES

“Acute Radiation Syndrome: A Fact Sheet for Clinicians” CDC. Aeba et al., “Lazaroid U74500A as an additive to University of Wisconsin solution for pulmonary grafts in the rat transplant model,” J. Thorac. Cardiovasc. Surg., 1992; 104 (5): 1333-9.
Bankura and Chandra, “Hydroxide Ion Can Move Faster Than an Excess Proton through One-Dimensional Water Chains in Hydrophobic Narrow Pores,” J. Phys. Chem. B, 2012; 116:9744-9757.
Becker, “New concepts in reactive oxygen species and cardiovascular reperfusion physiology,” Card. Res., 2004; 61:461-470.
“Biological effects of radiation,” Reactor Concepts Manual, USNRC Technical Training Center. Chaplin, “Oxygen and water,” 2022.
Check and Hunter, “The Scientific Basis for the Biologic Activities of RheothRx.™. Copolymer: A Rheologic, Antithrombotic and Cytoprotective Preparation,” CytRx Corp., 1988.
Choudhary et al., “Synthesis, characterization and antioxidant activity of some transition metal complexes with terpenoid derivatives,” J. Chil. Chem. Soc. 2011; 56 (4): 911-7.
Citrin and Mitchell, “Mechanisms of normal tissue injury from irradiation,” Semin. Radiat. Onco., 2017; 27:316-324.
Coghlan et al., “Allopurinol pretreatment improves postoperative recovery and reduces lipid peroxidation in patients undergoing coronary artery bypass grafting,” J. Thorac. Cardiovasc. Surg., 1994; 107:248.
Codorniu-Hernandez and Kusalik, “Mobility Mechanism of Hydroxyl Radicals in Aqueous Solution via Hydrogen Transfer,” J. Am. Chem. Soc., 2012; 134 (1): 532-538.
Cosenza et al., “Protective effect of the lazaroid U74006F in cold ischemia-reperfusion injury of the liver,” Hepatology, 1994; 19:418-425
Desouky et al., “Targeted and non-targeted effects of ionizing radiation,” J. Radiat. Res. Appl. Sci., 2015; 8 (2): 247-254.
Euler, “Role of oxygen-derived free radicals in canine reperfusion arrhythmias,” Am. J. Physiol., 1995; 268: H295
Flaherty et al., “Recombinant human superoxide dismutase (h-SOD) fails to improve recovery of ventricular function in patients undergoing coronary angioplasty for acute myocardial infarction,” Circulation, 1994; 89:1982.
Flaherty, “Myocardial injury mediated by oxygen free radicals,” Am J. Med., 1991; 91: 79S.
Granger et al., “Ischemia reperfusion injury: role of oxygen-derived free radicals,” Acta Physiol. Scand., 1986; 47: S548.
Hall et al., “21-Aminosteroid lipid peroxidation inhibitor U74006F protects against cerebral ischemia in gerbils,” Stroke, 1988; 19:997-1002.
Han et al., “H2S responsive PEGylated poly(lipoic acid) with ciprofloxacin for targeted therapy of Salmonella,” J. Control. Release, 2022; 351:896-906.
Hannig et al., “Poloxamine 1107 sealing of radiopermeabilized erythrocyte membranes,” Int. J. Radiat. Bio., 1999; 75:379-385.
Hannig et al., “Surfactant sealing of membranes permeabilized by ionizing radiation,” Radiat. Res., 2000; 154:171-177.
Katz et al., “Improved small intestinal preservation after lazaroid U74389G treatment and cold storage in University of Wisconsin solution,” Transplantation, 1995; 59:694-8.
Kiang et al., “Lipid peroxidation after ionizing irradiation leads to apoptosis and autophagy,” Lipid Peroxidation, 2012.
Kostova and Balkansky, “Metal complexes of biologically active ligands as potential antioxidants,” Curr. Med. Chem., 2013; 20 (36): 4508-39.
Land et al., “The beneficial effect of human recombinant superoxide dismutase on acute and chronic rejection events in recipients of cadaveric renal transplants,” Transplantation, 1994; 57:211.
Lee, “Cytoprotection by stabilization of cell membranes,” Ann. N.Y. Acad. Sci., 2002; 961:271-275.
Levitt et al., “Reduction of infarct size during myocardial ischemia and reperfusion by lazaroid U-74500A, a nonglucocorticoid 21-aminosteroid,” J. Cardiovasc. Pharmacol., 1994; 23:136-140.
Liu et al., “Protective role of trehalose during radiation and heavy metal stress in Aureobasidium subglaciale F134,” Sci. Rep., 2017; 7:17586.
Matros et al., “Sugars as hydroxyl radical scavengers: proof-of-concept by studying the fate of sucralose in Arabidopsis,” The Plant J. 2015; 82:822-839.
Nalini et al., “Oxygen free radical induced damage during intestinal ischemia/reperfusion in normal and xanthine oxidase deficient rats,” Mol Cell. Biochem., 1993; 124:59-66.
Park et al., Arch. Surg., 1994; 129:857-60.
Parks et al., “Ischemic injury in the cat small intestine: role of superoxide radicals,” Gastroenterology, 1982; 82:9-15.
Pollack et al, “A randomized double-blind trial of the use of human recombinant superoxide dismutase in renal transplantation,” Transplantation, 1993; 55:57-60.
Rashid and William-Olsson, “Influence of allopurinol on cardiac complications in open heart operations,” Ann. Thorac. Surg., 1991; 52:127-30.
Singh and Seed, “BIO 300: a promising radiation countermeasure under advanced development for acute radiation syndrome and the delayed effects of acute radiation exposure,” Expert Opin. Investig. Drugs.,2020; 29 (5): 429-441.
Seed et al., “Medical countermeasures for unwanted CBRN exposures: part II radiological and nuclear threats with review of recent countermeasure patents,” Expert Opin. Therapeutic Patents., 2016; 26:12, 1399-1408.
Shackleton et al., “Effect of a 21-aminosteroid, U74006F, on lipid peroxidation and glomerulotubular function following experimental renal ischemia,” J. Surg. Res., 1994; 57:433-7.
Smart and Shea, “Galactic cosmic radiation and solar energetic particles,” Handbook of Geophys., 1985.
Soneru et al., “Mg ATP and Antioxidants Augment the Radioprotective Effect of Surfactant Copolymers,” Health Phys., 2011; 101 (6): 731-738.
Stone et al. Am. J. Vet. Res., 1992; 53:2153-6.
Tale et al., “Trehalose-funtionalized block copolymers form serum-stable micelles,” Polym. Chem., 2014; 5:5160-5167.
Terry et al., “Oxidative cell membrane alteration: evidence for surfactant-mediated sealing,” N.Y. Acad. Sci., 1999; 888:274-284.
Timoshnikov et al., “Mechanistic insights of chelator complexes with essential transition metals: antioxidant/pro-oxidant activity and applications in medicine,” Int. J. Mol. Sci., 2022; 23 (3): 1247.
Van Ye et al., “Inhibition of intestinal lipid peroxidation does not minimize morphologic damage,” J. Surg. Res., 1993; 55:553-8.
Vanhaecke et al., “Effect of superoxide dismutase on infarct size and postischemic recovery of myocardial contractility and metabolism in dogs,” J. Am. Coll. Cardio., 1991; 18:224
Weng et al., “Effects of Water on Structure and Dynamics of Trehalose Glasses at Low Water Contents and its Relationship to Preservation Outcomes,” Sci. Rep., 2016; 6:28795.
Yang et al., “PEGylated poly(alpha-lipoic acid) loaded with doxorubicin as a pH and reduction dual responsive nanomedicine for breast cancer therapy,” Biomacromolecules, 2018; 19 (11): 4492-503.
Yasuda et al., “Dystrophic heart failure blocked by membrane sealant poloxamer,” Nature, 2005; 436:1025-9.
Yoshinaga et al., “Protection by Trehalose of DNA from Radiation Damage,” Biosci. Biotech. Biochem., 61 (1): 160-161.
   U.S. Pat. Nos.
   5,725,839;
   5,696,109;
   5,691,360;
   5,683,982;
   5,659,055;
   5,659,049;
   5,648,377;
   5,646,149;
   5,643,943 and
   5,623,052.

What is claimed is:

1. A method for treating effects of ionizing radiation in a subject in need thereof, the method comprising administering to the subject in need thereof a therapeutic composition comprising a surfactant copolymer and a pharmaceutically acceptable carrier.

2. The method of claim 1, wherein the method further comprises administering to the subject an antioxidant, a cellular energy source, or any combination thereof.

3. The method of claim 1, wherein the surfactant copolymer comprises one or more of the following: a polyalkyl, a polyglycerol, a saccharide, a poloxamer, a meroxapol, a poloxamine, a tetronic, a PLURADOT™ polyol, or any combination thereof.

4. The method of claim 1, wherein the surfactant copolymer is comprised of one or more amphiphilic block copolymers.

5. The method of claim 4, wherein a hydrophobic block of the amphiphilic block copolymer is comprised of a hydrophobic polypeptide, polyoxypropylene, polystyrene, polyglycolide, polylactide, poly(lactic-glycoacid), polycaprolactone, hydrophobic polyurethane, polyester, poly-N-isopropylacrylamide, polymethylmethacrylate, poly(2-dimethylaminoethylmethacrylate), polyethylene, polypropylene, polyisoprene, polybutylene, polybutadiene, poly(styrene-butadiene), polyvinyl chloride, polytetrafluoroethylene, polydimethylsiloxane, or any combination thereof.

6. The method of claim 4, wherein a hydrophilic block of the amphiphilic block copolymer is comprised of polyethylene oxide, polyvinyl alcohol, hydrophilic polyurethane, polyvinylpyrrolidone, polyacrylamide, polyacrylic acid, poly(meth)acrylic acid, polyethylenimine, poly(methyl vinyl ether), poly(styrene-maleic anhydride), polyethylene glycol ether, polyamine, a hydrophilic polypeptide, or any combination thereof.

7. The method of claim 4, wherein the surfactant copolymer is synthesized from one or more hydrophilic blocks and one or more hydrophobic blocks.

8. The method of claim 1, wherein:
the surfactant copolymer is comprised of surfactants which have been purified to mitigate hydroperoxides and low molecular weight fragments;
the surfactant copolymer is comprised of purified or chemically modified poloxamer 188 (P188); and/or
the amount of the surfactant copolymer in the blood ranges from 0.005 to 20.0 mg/ml.

9. The method of claim 2, wherein
the antioxidant is comprised of ascorbic acid, tocopherol, Vitamin A, mannitol, a bioflavonid, a flavonoid, a flavone, a flavonol, proanthocyanidin, selenium, gluthathione, N-acetyl-cysteine, superoxide dismutase, lipoic acid, coenzyme Q-10, beta-carotene, lycopene, lutein, polyphenol, BHA, BHT, surfactant oxidants, or any combination thereof;
the antioxidant is comprised of transition metal ions;
the antioxidant includes one or more of the following metals: Ag, Au, Zn, Fe, Mn or any combination thereof;
the transition metal functions as a moiety of a larger molecular complex;
the antioxidant is N-acetyl-cysteine and is administered in an amount ranging from 25 mg to 1000 mg;
the antioxidant is lipoic acid and is administered in an amount ranging from 0.02 to 4.2 mg/ml in the blood volume;
the cellular energy source is comprised of adenosine triphosphate, adenosine diphosphate, phosphocreatine, phosphocreatine or some combination thereof;
the cellular energy source is R-ATP in a dose ranging from 0.1% to 2.0% w/v; and/or
trehalose in a dose range of 0.1 to 20 mM is added as a cofactor.

10. The method of claim 1, wherein the composition is administered to the subject after radiation exposure, preferably up to 48 hours after radiation exposure; and further preferably for up to five days after radiation exposure.

11. The method of claim 1, wherein the composition is administered to the subject prior to ionizing radiation exposure, preferably up to 24 hours prior to radiation exposure.

12. The method of claim 1, wherein the therapeutic composition is administered via intravascular, intramuscular, subcutaneous, intrathecal or intraperitoneal injection to the site of radiation injury.

13. The method of claim 1, wherein administering the therapeutic composition has one or more of the following effects:
increases cell viability by restoring ionic barrier function to cell membranes damaged by ionizing radiation;
decreases radical oxygen intermediate (ROI) generation following exposure of mammalian cells to ionizing radiation;
inhibits the cellular unfolded protein response activated by radiation damage;
increases viability of irradiated cells by a statistically significant proportion, preferably by at least 40% at a time 48 hours subsequent to the exposure event, compared to untreated cells; and/or
causes a statistically significant decrease, preferably by at least 10%, in tissue inflammation after radiation exposure compared to untreated patients.

14. The method of claim 1, wherein the dosage of radiation inflicts acute radiation syndrome (ARS), gastrointestinal radiation syndrome (GIARS), delayed effects of acute radiation exposure (DEARE), or any combination thereof.

15. The method of claim 1, wherein the dosage of radiation inflicts radiation dermatitis.

16. The method of claim 1, wherein the radiation is inflicted via radiation therapy, long term exposure due to radiation contamination, cosmic radiation, nuclear detonation, or any combination thereof.

17. The method of claim 1, wherein the radiation dosage is inflicted by alpha particles, beta particles, neutron particles, electromagnetic radiation, or any combination thereof.

18. The method of claim 17, wherein the electromagnetic radiation occurs in the form of X-rays, gamma rays, ultraviolet rays, or any combination thereof.

19. The method of claim 1, wherein the pharmaceutically acceptable carrier is one or more of the following: a dispersion media: an antibacterial or antifungal agent; isotonic; an absorption delaying agent, or any combination thereof.
